(19) United States
(12) Patent Application Publication
(10) Pub. No.: US 2004/0034323 A1
(43) Pub. Date: Feb. 19, 2004

(54) Device for retaining a syringe and needle guard for same

(76) Inventor: Wilfried Manthey, Zornheim (DE)

Correspondence Address: COLLARD & ROE, P.C., 1077 Northern Boulevard, Roslyn, NY, 11576-1696, US

(21) Appl. No.: 10/223,574

(22) Filed: Aug. 19, 2002

(51) Int. Cl. A61M 5/32

(52) U.S. Cl. 604/198

(57) ABSTRACT

The invention relates to a device (1) for receiving and retaining at least one syringe body (2) of an injection syringe (3), co-operating with an injection cannula and a syringe plunger, having a cylindrical-type casing (4) forming a housing chamber for the syringe body (2) at least partially enclosing the syringe body (2) and being bounded by a first and a second end face in the direction of its longitudinal extension and being provided with positive and/or friction fit retaining means for the injection syringe (3). The casing (4) is provided with a longitudinal slit (5) extending across a greater part of its length for receiving a syringe body (2) joined to the injection cannula.

### BACKGROUND OF THE INVENTION

1. Field of the Invention

The invention relates to

a device for receiving and retaining at least one syringe body of a syringe, which co-operates with a an injection cannula and a syringe plunger, with a casing forming a cylinder-type housing chamber for the syringe body, which at least partially encloses the syringe body and is bounded by a first and a second end face in the direction of its longitudinal extension and provided with positive and/or friction-fit retaining means for the syringe,

and a needle guard for syringes with a tubular protective casing for enclosing the syringe body of the syringe, at least around the greater part of its circumference, the protective casing being provided with a fixing element for fixing the syringe body in its relative position.

2. The Prior Art

Various devices for receiving and retaining at least one syringe body of a syringe are already known—WO 87/02254 A—in which a casing of a cylindrically shaped housing chamber is closed off by an end wall in the region of one end face and the casing has only one opening through which to pass the syringe cannula, whilst its other end face is open so that a syringe body of a syringe can be pushed through the full cross section. To use the syringe in this receiving and retaining device, the syringe is pushed in the longitudinal direction of the cylindrical housing chamber, but as the injection cannula is inserted through the bore in the end wall at one end, the injection cannula can become damaged or the operating personnel can be injured during pushing through the opening when withdrawing the injection cannula from the housing chamber.

Devices are also known, whereby the needle of a syringe can be enclosed so as to prevent injury to the operating personnel. Not only do injection needles pose a risk of injury after use, they also risk causing infections. In order to prevent such infections caused by injection needles which have been in contact with blood, bodily secretions or parts of the patient's tissue, a number of devices have been proposed which enable the needle tip of the used injection needle to be securely covered or enclosed. When such devices, in the form of a protective cylinder which is pushed over the syringe body for example, are in the position in which they cover the injection needle, they are fixed only to the foremost region, i.e. the end of the syringe body directed towards the needle. Such devices offer only a certain degree of safety because the needle and the syringe body can be only partially and not totally enclosed. This means that the now unprotected syringe body is at risk of breaking and there is always a risk that the connection between protective sleeve and syringe body may be faulty.

### SUMMARY OF THE INVENTION

The underlying objective of the present invention is to propose a device for receiving and retaining a syringe body of a syringe, in which the risk of injury when inserting the syringe in the housing chamber is as low as possible and affords a stable hold of the syringe. Yet another objective is to propose a needle guard for syringes, which, when in its position covering the needle tip, encloses the entire system comprising needle and syringe body and the fixing elements of which are particularly strong and secure, preventing faulty operation

The objective is achieved by the invention due to the fact that the casing for receiving a syringe body connected to the injection cannula is provided with a longitudinal slit extending across a major part of its length. The advantage of this solution is that syringes, in particular the syringe body thereof, may be made with thin wall thicknesses and optionally also out of glass because by using the receiving and retaining device, the forces needed to administer the injection can be transmitted via the receiving and retaining device, which has the added advantage of ruling out breakage, especially of the syringe body, if a syringe of this type is dropped. Furthermore, it is actually much easier to insert the syringe in this housing chamber because the latter is provided with a longitudinal slit across the greater part of its length, thereby further reducing the risk of damage to the injection cannula and injury to the user.

In another embodiment of the device, a receiving part is provided for the injection flange of the syringe on the first end face of the casing, in particular as an integral piece with the casing, the advantage of which is that by providing a special receiving part for the injection flange of the syringe, the latter can also be fixed to prevent any sliding in the axial direction.

The embodiment of the device in which the casing is closed around its entire circumference in the area around part of its second face, preferably in the range of 2 to 12% of the length of the casing, provides a design whereby the syringe body is positively enclosed and fixed at its needle-side end, preventing it from slipping out of the device due to the forces applied when using the syringe.

Some embodiments of the device are advantageously designed to enclose the syringe body in order to prevent breakage or damage caused by objects which are at least bigger than the lateral longitudinal slit of the casing. The fact that the surface shape of the internal surface of the casing is adapted to a casing surface of the syringe, provides direct support for the syringe across the greater part of its external surface, which significantly improves the mutual fixing of the two parts to one another.

Also of advantage are the embodiments in which the internal dimensions of the casing cross section are slightly smaller than the external dimensions of the cross section of the syringe body and the internal dimensions of the receiving part are slightly smaller than the external dimensions of the injection flange, because the elastic deformation needed to deform the casing generates a pre-tensioning force in the casing and syringe body, resulting in an improved retaining force.

In another embodiment of the device, the distance between the edges of the longitudinal slit is smaller than the smallest cross-sectional extension of the syringe body, the advantage of which is that when the syringe is inserted, the syringe body has to be placed in the casing by a snap-fit action, so that the syringe is enclosed in a positive arrangement and positively fixed.

In one embodiment, the internal surface of the casing and/or an internal surface of the receiving part has a surface structure and/or an increased surface roughness, which improves static friction, thereby increasing the amount of force needed to remove the syringe from the casing, which also improves the safety of the mounting.

Another advantage is the fact that other sleeves can be attached to the casing either for operational purposes or as a protection. Another possibility offered by the retaining device is that the injection syringe integrated in the device can be attached to apparatus, of the type which might be used to automate manipulation of injection syringes, for example. Since the retaining device is provided as a groove, push-on sleeves can be fixed to the casing in different axial positions.

Also of advantage is another embodiment, in which the receiving part has a widening intended as a gripping plate extending at least almost radially outwards in a plane perpendicular to the longitudinal extension of the casing, since providing a gripping plate provides a larger support surface for the fingers than the syringe flange during usage, which significantly improves operating safety, especially in the case of small syringes.

However, the objective is also achieved by the invention independently of the above by means of a needle guard for an injection syringe with a tubular protective casing for enclosing the syringe body of the injection syringe, at least across a greater part of its circumference, the protective casing being provided with a fixing element to fix the syringe body in its relative position. This needle guard has a receiving and retaining device as proposed by the invention and an internal diameter that is larger than the external diameter of the casing of the receiving and retaining device. The fixing element is provided in the form of a retaining mechanism which co-operates with the retaining mechanism provided on the external surface of the receiving and retaining device. The advantage of this is that a better protective effect can be obtained because the injection system is no longer able to accidentally fall out of the housing chamber in which a longitudinal slit is provided.

Also of advantage is another embodiment in which the receiving and retaining device can be arranged between the injection syringe and the protective casing because this means that protective casings of the same construction can be used for different types of syringe. In effect, it is merely necessary to adapt the construction of the receiving and retaining device to the syringe type, which offers a significant economic advantage overall.

The embodiment of the needle guard in which the protective casing has at least one guide sleeve and/or at least one needle-protecting sleeve offers an advantage insofar as a part of the protective casing can remain in its original position enclosing the syringe body whilst another part of the protective casing can be pushed in the direction of the needle tip in a position in which it encloses the entire needle, including its tip, after use.

Also of advantage is the embodiment of the needle guard in which the guide sleeve at least partially encloses the receiving and retaining device in a plane perpendicular to its longitudinal extension, which, firstly, enables the injection syringe body to be enclosed in a positive arrangement preventing the injection syringe from falling out and, secondly, protects the syringe body against damage from any objects which might get into it.

By virtue of the advantageous embodiment of the needle guard in which in an internal surface and/or an external surface of the guide sleeve is provided in the form of a circular cylinder casing surface, the guide sleeve and the receiving and retaining device are rotatable relative to one another. Moreover, the guide sleeve and another sleeve mounted thereon are also rotatable relative to one another. Another advantage is the fact that sleeves of a circular cylindrical shape are much more rigid than other cross-sectional shapes.

Also of advantage is the embodiment of the needle guard in which the guide sleeve has a lateral opening extending across at least a part of its length at least in the direction of its longitudinal extension , which reduces the amount of force needed to push on the receiving and retaining device. It is of advantage to use guide sleeves fitted on a retaining device operated by an elastic expanding action in the region of the retaining device and having lateral openings in the region of the retaining device because the retaining device can be opened or closed more easily.

The advantageous embodiments in which the guide sleeve has at least one retaining mechanism on its internal surface or is joined to the holding and retaining device in a positive and/or friction fit enable the guide sleeve to be securely fixed on the receiving and retaining device in a predetermined position relative to one another.

Also of advantage is an embodiment in which the guide sleeve has at least one retaining mechanism on its external surface, which enables another sleeve to be fixed onto the guide sleeve.

The advantage of the needle guard in which the guide sleeve is at least partially enclosed in a plane perpendicular to the longitudinal extension of the guide sleeve is that the overall structure of the device is much stronger and more reliable due to the additional needle-protecting sleeve.

The embodiments of the needle guard in which an internal surface and/or an external surface of the needle-protecting sleeve has a circular cylindrical casing surface offer an advantage because the needle-protecting sleeve can be placed on a guide sleeve with a circular cross section so as to be rotatable and slideable in the axial direction.

Also of advantage is an embodiment in which the needle guard has a lateral opening extending in the direction of its longitudinal extension, at least across a part of its length, because the force needed to push the needle guard onto the receiving and retaining device is reduced. The advantage of needle-protecting sleeves fitted with a retaining mechanism operated by elastic expansion of the needle guard in the region of the retaining mechanism and having lateral openings in the region of the retaining mechanism is that the retaining device can be opened and closed more easily.

By virtue of the embodiments of the needle guard in which the needle-protecting sleeve has at least one retaining mechanism on its internal surface or is joined to the guide sleeve in a positive and/or friction fit is that the needle-protecting sleeve can be fixed in predetermined positions relative to the injection syringe.

Advantage is also to be had from an embodiment in which the needle-protecting sleeve and the guide sleeve overlap with one another in the axial direction, at least across a length corresponding to once the diameter of the guide sleeve, because the mutual overlap of the needle-protecting sleeve and the guide sleeve makes the connection between the two sleeves stronger and more stable.

Finally, the embodiment of the needle guard in which the needle-protecting sleeve extends beyond the needle tip by one of its end regions in the direction of the longitudinal extension of the injection syringe, at least in one of its fixable positions, and covers the needle tip is of advantage because the fact that the needle tip is covered after the injection syringe has been used protects the user against injury and infection from the injection cannula.

The embodiment of the needle guard in which the needle-protecting sleeve has a cover on one of its ends offers the advantage of increased protection, because additionally covering the front opening of the needle-protecting sleeve also prevents penetration of this opening by fingers, particularly of children, which could otherwise cause injury due to the needle tip.

The advantage of the needle guard in which the cover is designed so that it is encased by the cylinder casing surfaces enclosing the needle-protecting sleeve when in the open position is that in the state prior to using the injection syringe, the cover lies against the external surface of the guide sleeve and thus has no parts projecting beyond the external cylindrical shape of the needle-protecting sleeve as this would otherwise hamper manipulation of the injection syringe.

The advantage of the embodiment of the needle guard in which the cover is integrally joined to the needle-protecting sleeve by means of an elastically deformable connecting point is that it obviates the need for an additional connecting piece to fix the cover. If provided as an injection moulded part, the needle-protecting sleeve with integrated cover can be produced in a single work process.

The design of the needle guard in which the cover is provided in the form of a first cover lobe and a second cover lobe offers an advantage because the cover lobes close in a roof-shaped arrangement when the needle-protecting sleeve is in the closed state. This makes the external shape of the needle guard as a whole much more compact.

The advantage of the embodiments of the needle guard in which a first retaining mechanism is provided on the end of the cover lobe and a second retaining mechanism is provided on the second cover lobe or the first cover lobe and the second cover lobe are joined in a positive and/or friction fit by means of the retaining mechanisms is that the cover can be safely closed by the retaining mechanisms.

The advantage of the embodiment of the needle guard in which, when the needle guard is in at least one of the fixable positions, the cover is partially or totally covered when the needle tip is in a closed position, is that once the injection needle has been used, the needle tip is covered so that the needle tip can not come into contact with and injure any parts of the body. In particular, there is also ensures that there is no possibility of children, for example, putting their fingers through the front opening of the needle-protecting sleeve and injuring themselves on the needle tip.

### BRIEF DESCRIPTION OF THE DRAWINGS

The invention will be explained in more detail below with reference to examples of embodiments illustrated in the appended drawings, which provide simplified diagrams of:

FIG. 1 a device for receiving and retaining a syringe body of an injection syringe;

FIG. 2 a receiving and retaining device with injection syringe;

FIG. 3 a guide sleeve;

FIG. 4 a needle-protecting sleeve;

FIG. 5 a section through a needle-protecting sleeve along line V—V indicated in FIG. 4;

FIG. 6 a detail of the cover lobe of the needle-protecting sleeve;

FIG. 7 a detail of the cover lobe of the needle-protecting sleeve;

FIG. 8 the two cover lobes joined to one another by their retaining mechanisms;

FIG. 9 a two-part protective casing (pre-assembled);

FIG. 10 a needle guard with injection syringe prior to use;

FIG. 11 a detail of the needle guard illustrated in FIG. 10;

FIG. 12 a needle guard with injection syringe after use.

### DETAILED DESCRIPTION OF PREFERRED EMBODIMENTS

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc,. relate to the drawing specifically being described and can be transposed in terms of meaning to a new position when another position is being described. Individual features or combinations of features from the different embodiments illustrated and described may be construed as independent inventive solutions or solutions proposed by the invention in their own right.

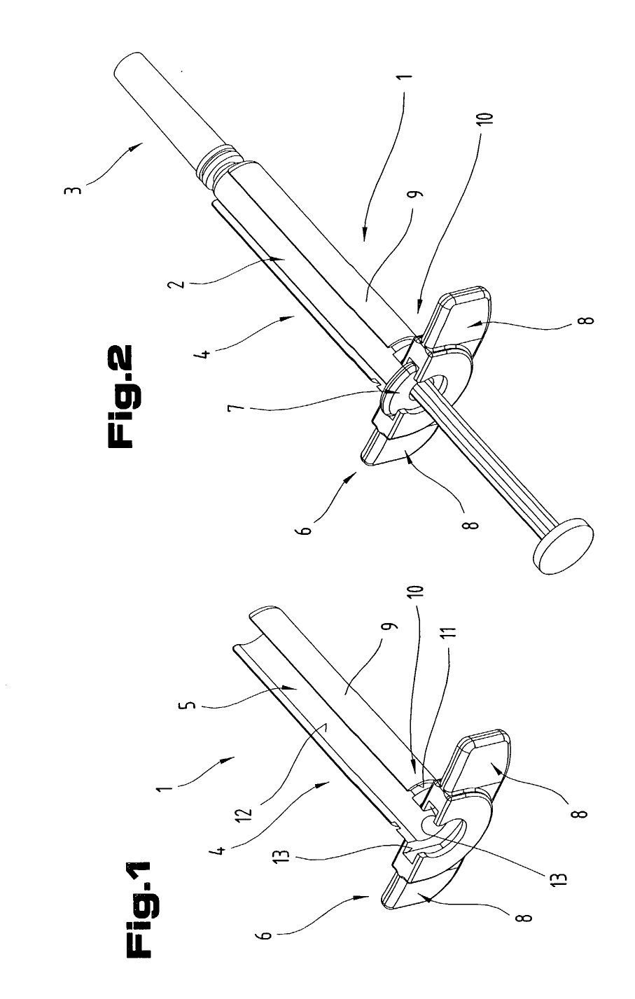

FIGS. 1 and 2 illustrate a device 1 as proposed by the invention for receiving and retaining a syringe body 2 of an injection syringe 3 without and with the injection syringe 3 attached. Such a device might be used with ready-filled, ready-to-use glass syringes, for example.

At its front region, the device has a circular cylindrically shaped casing 4, with a longitudinal slit 5 extending across its entire length. However, the casing 4 may also have a serni-circular or more or less U-shaped cross section. At its rear region, the device 1 has a receiving part 6 for receiving and retaining a syringe flange 7. The receiving part 6 is widened at the sides by means of gripping plates 8, on which the fingers rest when the syringe is being used. On its external surface 9, the casing 4 is provided with a retaining mechanism 10, provided in the form of a radially extending groove 11. Another push-on sleeve can be fixed on this retaining mechanism 10. Alternatively, however, the retaining mechanism 10 may also be used as a means of attaching the receiving and retaining device to an injection syringe 3, for example, in a device designed to permit automated manipulation of injection syringes.

The width of the longitudinal slit 5 of the receiving and retaining device is slightly smaller than the external diameter of the syringe body. Placing the injection syringe 3 in the receiving and retaining device 1 therefore causes a reversible elastic deformation and once this has been overcome, the syringe body 2 is held fixed in the device in a positive fit. Alternatively, the injection syringe 3 can be fixed in a different manner, in which case the internal diameter and the internal dimensions of the cross section of the casing 4 and the receiving part 6 are slightly smaller than the corresponding cross-sectional dimensions of the syringe body 2 and the syringe flange 7 so that inserting the injection syringe 3 leads to a residual elastic deformation. The injection syringe 3 is retained in a friction fit due to the resultant forces. An even stronger retaining force can be obtained due to the fact that an internal surface 12 of the casing 4 and/or an internal surface 13 of the receiving part 6 has a surface structure or a surface roughness, which increases the static friction accordingly. Another way of increasing the reliability of the mounting is to adapt the shape of the internal surface 12 of the casing 4 to the casing surface of the injection syringe 3. Another option in the region of the front end of the casing 4 is to provide the latter with a totally closed circumference along at least a part, preferably 2 to 12%, of its length.

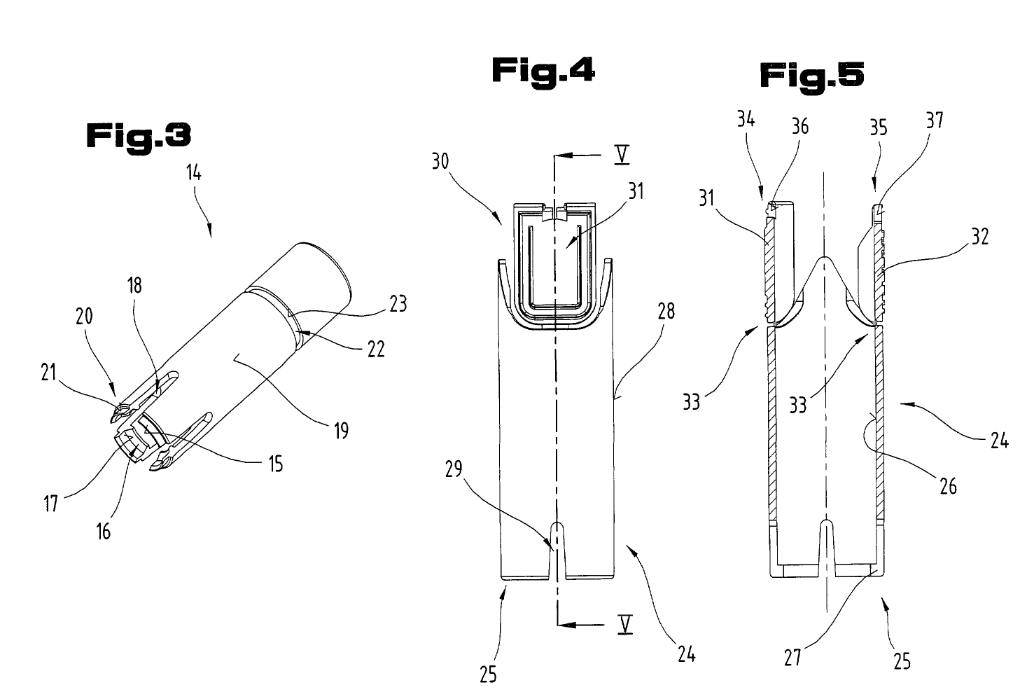

FIG. 3 illustrates a circular, cylindrically shaped guide sleeve 14. On an internal surface 15, this guide sleeve 14 has a retaining mechanism 16, provided in the form of a snap-fit bead 17. Lateral openings 18 in the guide sleeve 14 extending in the axial direction facilitate operation of the snap-fit bead 17 due to elastic deformation. On an external surface 19, the guide sleeve 14 has a retaining mechanism 20 in the form of a stop 21 and another retaining mechanism 22 in the form of a groove 23 extending radially across the entire circumference 23.

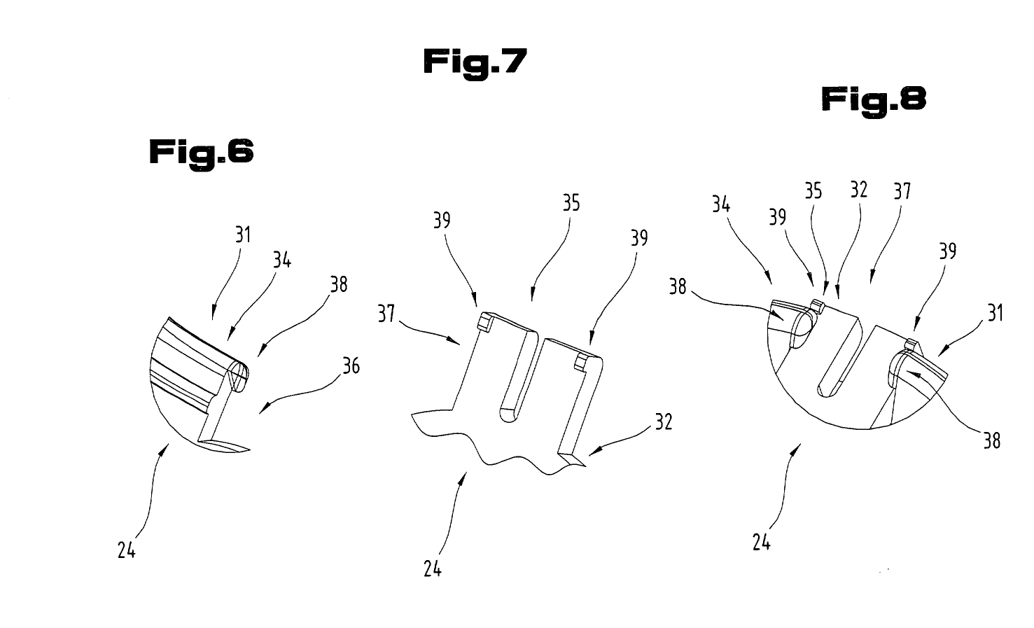

FIGS. 4 to 8 illustrate a circular, cylindrically shaped needle-protecting sleeve 24 with a retaining mechanism 25 on an internal surface 26, in the form of a snap-fit bead 27. The needle-protecting sleeve 24 may have a circular, cylindrical structure both on the internal surface 26 and on an external surface 28 or may be of any other—generally—cylindrical shape. Operation of the snap-fit bead 27 by elastic deformation is facilitated by lateral openings 29 in the needle-protecting sleeve 29 extending in the axial direction. At the end of the retaining mechanism 25 opposite the needle-protecting sleeve 24 is a cover 30, comprising a first cover lobe 31 and a second cover lobe 32. The cover lobes 31, 32 are integrally joined to the needle-protecting sleeve 24. A connecting point 33 of the first cover lobe 31 and a connecting point 33 of the second cover lobe 32 are designed with a slimmer wall thickness in the form of an integral hinge so that the cover lobes 31, 32 can be pivoted by elastically deforming the material at the connecting points 33. The cover lobes 31, 32 are illustrated in their basic position in FIGS. 4 and 5. Their cross-sectional shape relative to the longitudinal extension of the needle-protecting sleeve 24 is the same as the cross-sectional shape of the rest of the needle-protecting sleeve 24. In other words, they are contained by common enclosing circular cylindrical surfaces. At their front, free ends, the first cover lobe 32 has a retaining mechanism 35, by means of which the ends of the two cover lobes 31, 32 are joined to one another in a friction and/or positive fit. The retaining mechanism 34 of the first cover lobe 31 is provided in the form of a recess 36 and the retaining mechanism 35 of the second cover lobe 32 is provided by means of a tongue 37 which fits in the retaining mechanism 34. At the foremost end of the recess 36, the retaining mechanism 34 has a respective inwardly extending catch 38 (FIG. 6). At the foremost end of the tongue 37, the retaining mechanism 35 has a respective outwardly extending catch 39 (FIG. 7). The first cover lobe 31 and the second cover lobe 32 are connected by pushing the tongue 37 of the retaining mechanism 35 into the recess 36 of the retaining mechanism 34, the catches 38 having to be overcome by elastic deformation (FIG. 8).

However, any friction and/or positive catch and/or snap-fit connection known from the prior art may be used to connect the first cover lobe 31 to the second cover lobe 32.

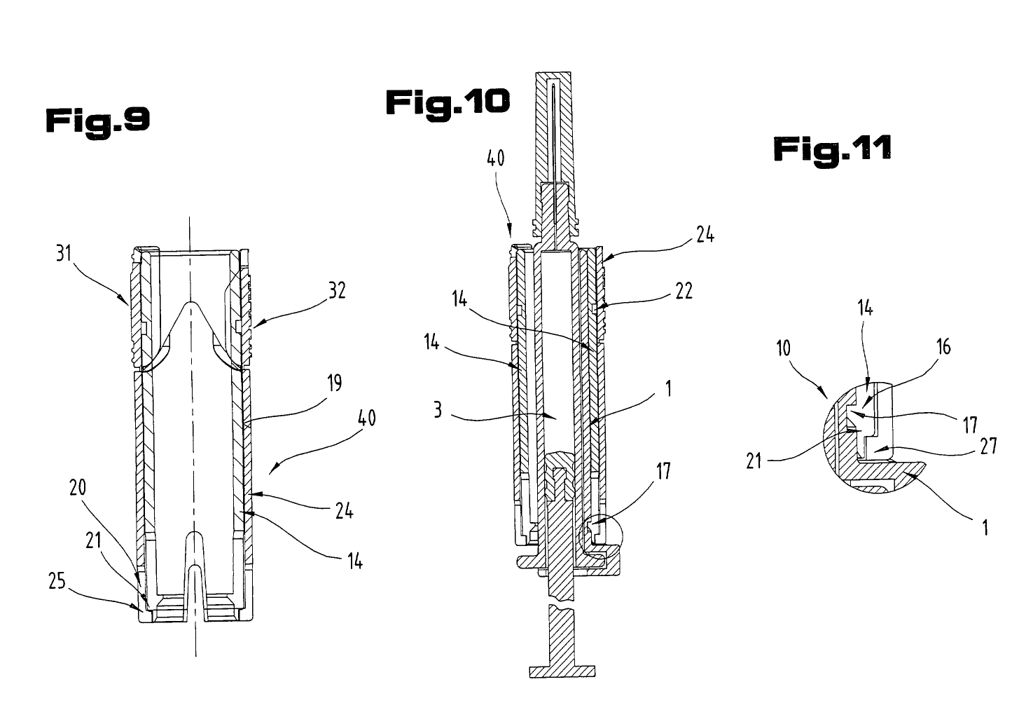

FIG. 9 illustrates a protective casing 40 consisting of the guide sleeve 14 and the needle-protecting sleeve 24. The retaining mechanism 25 of the needle-protecting sleeve 24 is latched on the stop 21 of the retaining mechanism 20 of the guide sleeve 14. The first cover lobe 31 and the second cover lobe 32 are in their initial position, in which they sit against the external surface 19 of the guide sleeve 14.

FIGS. 10 to 12 illustrate a needle guard with an injection syringe 3 fitted before and after use of the injection syringe 3. The snap-fit bead 17 of the retaining mechanism 16 of the guide sleeve 14 is located in the retaining mechanism 10 of the receiving and retaining device 1 (FIG. 11). The protective casing 40 comprising the guide sleeve 14 and the needle-protecting sleeve 24 clamped on it encloses the receiving and retaining device 1 with the injection syringe 3 integrated in it so that the injection syringe 3 is enclosed around its entire circumference.

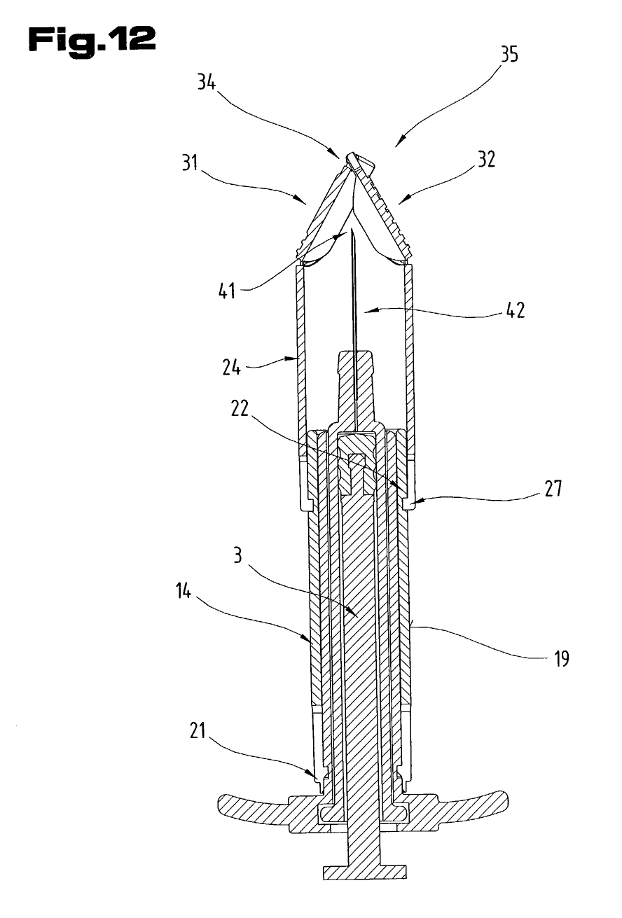

Once the injection syringe 3 has been used, the snap-fit bead 27 of the needle-protecting sleeve 24 is pulled back over the stop 21 of the guide sleeve 14 and pushed so that it slides axially along the external surface 19 of the guide sleeve 14 in the direction of the needle tip 41 until the snap-fit bead 27 of the needle-protecting sleeve 24 locates in the retaining mechanism 22 of the guide sleeve 14 (FIG. 12). In a subsequent step, the two cover lobes 31, 32 are then bent inwards by the user to close off the front opening of the needle-protecting sleeve 24. Having been bent inwards, the first cover lobe 31 and the second cover lobe 32 are then fixedly connected to one another at the tip by means of their retaining mechanisms 34, 35. In this position, the injection syringe 3 is additionally enclosed and protected around its entire circumference, whilst the injection cannula 42 is enclosed by the needle-protecting sleeve 24, and the two cover lobes 31, 32 joined to one another by their ends are covered in the axial direction, thereby preventing injury and infection by the injection cannula 42.

For the sake of good order, it should be pointed out that in order to provide a clearer understanding of the structure of the receiving and retaining device 1, it and its constituent parts are illustrated to a certain extent out of scale and/or on a larger scale and/or on a smaller scale.

The tasks underlying the independent solutions proposed by the invention may be taken from the description.

Above all, the individual embodiments of the subject matter illustrated in FIGS. 1, 2; 3; 4, 5, 6, 7, 8; 9; 10, 11, 12 constitute independent solutions proposed by the invention. The associated problems and solutions proposed by the invention may be found in the detailed descriptions of these drawings.

What is claimed is:

1. Device for receiving and retaining at least one syringe body of an injection syringe, co-operating with an injection cannula and a syringe plunger, having a cylindrical-type casing forming a housing chamber for the syringe body, which at least partially encloses the syringe body and is bounded in the direction of its longitudinal extension by a first and a second end face and is provided with positive and/or friction-fit retaining mechanism s for the injection syringe, characterised in that the casing for receiving a syringe body connected to the injection cannula is provided with a longitudinal slit extending across the greater part of its length.

2. Device as claimed in claim 1, characterised in that at least one receiving part for the syringe flange of the injection syringe is provided on the first end face of the casing as a holding means, in particular is integrally joined to the casing.

3. Device as claimed in claim 1, characterised in that the casing is closed around its entire circumference at least in the region of its second end face across a part, preferably in the region of 2 to 12%, of the length of the casing.

4. Device as claimed in claim 1, characterised in that the casing has an at least more or less U-shaped or semicircular cross section in the direction of its longitudinal extension.

5. Device as claimed in claim 1, characterised in that an internal surface of the casing and/or an external surface of the casing has a circular or arcuate cross section.

6. Device as claimed in claim 1, characterised in that the internal surface of the casing is adapted to the casing surface of the syringe body.

7. Device as claimed in claim 1, characterised in that the internal dimensions of the cross section of the casing are slightly smaller than the external dimensions of the cross section of the syringe body.

8. Device as claimed in claim 2, characterised in that the internal dimensions of the receiving part are slightly smaller than the external dimensions of the syringe flange.

9. Device as claimed in claim 1, characterised in that the distance between the edges of the longitudinal slit are slightly smaller than the smallest cross-sectional extension of the syringe body.

10. Device as claimed in claim 6, characterised in that the internal surface of the casing and/or an internal surface of the receiving part has a surface structure and/or an increased surface roughness.

11. Device as claimed in claim 1, characterised in that the casing has a retaining mechanism on its external surface for a pull-on sleeve serving as a protection for the needle.

12. Device as claimed in claim 11, characterised in that the retaining mechanism has one or more grooves extending at least across a part of the external surface of the casing.

13. Device as claimed in claim 12, characterised in that the groove or grooves extend or extends parallel with a plane perpendicular to the longitudinal extension of the casing.

14. Device as claimed in claim 2, characterised in that the receiving part is provided with a widening directed more or less radially outwards from the casing cross section serving as a gripping plate, in a plane perpendicular to the longitudinal extension of the casing.

15. Needle guard for injection syringes with a tubular protective casing for enclosing the syringe body of the injection syringe, at least around a major part of its circumference, the protective casing being provided with a fixing element for fixing the syringe body in its relative position, characterised in that a receiving and retaining device is provided as claimed in one of the preceding claims 1 to 14 and has an internal diameter which is bigger than the external diameter of the casing of the receiving and retaining device, and the fixing element is provided by a retaining mechanism which co-operates with the retaining mechanism disposed on the external surface of the casing of the receiving and retaining device.

16. Needle guard as claimed in claim 15, characterised in that the receiving and retaining device can be arranged between the injection syringe and the protective casing.

17. Needle guard as claimed in claim 15, characterised in that the protective casing has at least one guide sleeve and/or at least one needle-protecting sleeve.

18. Needle guard as claimed in claim 17, characterised in that the guide sleeve at least partially encloses the receiving and retaining device in a plane perpendicular to the longitudinal extension thereof.

19. Needle guard as claimed in claim 17, characterised in that an internal surface and/or an external surface of the guide sleeve is provided in the form of a circular cylindrical casing surface.

20. Needle guard as claimed in claim 17, characterised in that the guide sleeve has at least one lateral opening extending in the direction of its longitudinal extension, at least across a part of its length.

21. Needle guard as claimed in claim 17, characterised in that the guide sleeve has at least one retaining mechanism on its internal surface.

22. Needle guard as claimed in claim 17, characterised in that the guide sleeve is connected to the receiving and retaining device in a friction and/or positive fit.

23. Needle guard as claimed in claim 17, characterised in that the guide sleeve has at least one retaining mechanism on its external surface.

24. Needle guard as claimed in claim 17, characterised in that the needle-protective sleeve at least partially encloses the guide sleeve in a plane perpendicular to the longitudinal extension of the guide sleeve.

25. Needle guard as claimed in claim 17, characterised in that an internal surface and/or an external surface of the needle-protecting sleeve is provided in the form of a circular cylindrical casing surface.

26. Needle guard as claimed in claim 17, characterised in that the needle-protecting sleeve has a lateral opening extending at least in the direction of its longitudinal direction, at least across a part of its length.

27. Needle guard as claimed in claim 17, characterised in that the needle-protecting sleeve has at least one retaining mechanism on its internal surface.

28. Needle guard as claimed in claim 17, characterised in that the needle-protecting sleeve is connected to the guide sleeve in a friction and/or positive fit.

29. Needle guard as claimed in claim 17, characterised in that the needle-protecting sleeve and the guide sleeve overlap with one another in the axial direction at least across a length corresponding to once the diameter of the guide sleeve.

30. Needle guard as claimed in claim 17, characterised in that, in at least one of its fixable positions, the needle-protecting sleeve extends at one of its end regions beyond the needle tip in the direction of longitudinal extension of the injection syringe and covers the needle tip.

31. Needle guard as claimed in claim 17, characterised in that the needle-protecting sleeve has a cover at one end.

32. Needle guard as claimed in claim 31, characterised in that the cover is designed so that, when in the open position, it is contained by the cylindrical casing surfaces enclosing the needle-protecting sleeve.

33. Needle guard as claimed in claim 31, characterised in that the cover is integrally joined to the needle-protecting sleeve by means of an elastically deformable connecting point.

34. Needle guard as claimed in claim 31, characterised in that the cover is made up of a first cover lobe and a second cover lobe.

35. Needle guard as claimed in claim 34, characterised in that a first retaining mechanism is provided on the end of the first cover lobe and a second retaining mechanism is provided on the end of the second cover lobe.

36. Needle guard as claimed in claim 34, characterised in that the first cover lobe and the second cover lobe are connected in a friction and/or positive fit by means of the retaining mechanisms.

37. Needle guard as claimed in claim 31, characterised in that, when the needle-protecting sleeve is in at least one of the fixable positions, the cover partially or totally covers the needle tip in a closed position.
